(19) United States
(12) Patent Application Publication
(10) Pub. No.: US 2026/0215775 A1
(43) Pub. Date: Jul. 30, 2026

(54) AUTOMATIC MACHINE FOR ATTACHING SUTURES TO SURGICAL NEEDLES

(71) Applicants: Seyed Hasan ATYABI, Tehran (IR); Nakh Jarahan Teb Sina, Tehran (IR)

(72) Inventors: Seyed Hasan ATYABI, Tehran (IR); Mohammadreza SHEIKHI, Tehran (IR); Iman GHASEMI KAHRIZSANGI, Tehran (IR); Sayed Milad NABAVI RENANI, Tehran (IR); Mahmoud FARZIN, Isfahan (IR); Ala sadat ATYABI, Tehran (IR)

(21) Appl. No.: 19/144,678

(22) PCT Filed: Oct. 9, 2023
(86) PCT No.: PCT/IB2023/060108

§ 371 Date: Jun. 30, 2025

Related U.S. Application Data
(60) Provisional application No. 63/436,493, filed on Dec. 31, 2022.

(51) Int. Cl. A61B 17/06

(52) U.S. Cl. CPC A61B 17/06004

(57) ABSTRACT

A swaging apparatus for attaching a suture to a surgical needle comprising a suture guiding assembly, a needle management assembly, and a swaging assembly. The suture guiding assembly includes a suture gripping member comprising a first grip fixedly attached to a first jaw of a first parallel gripper through a fixed end of the first grip, and a second grip fixedly attached to a second jaw of the first parallel gripper through a fixed end of the second grip. The needle management assembly includes a needle securing module coaxially disposed opposite to the suture gripping member. The needle securing module comprises a second parallel gripper and a clamping member both having a reciprocating motion in an axis perpendicular to a longitudinal axis of the needle securing module. The swaging assembly comprises a first slider actuated by a first servo motor and a second slider actuated by a second servo motor.

### CROSS-REFERENCE TO RELATED APPLICATION

This application claims the benefit of priority from U.S. Provisional Patent Application Ser. No. 63/436,493, filed on Dec. 31, 2022, entitled “THE PROGRAMMABLE SWAGING MACHINE EQUIPPED WITH AUTOMATIC ALIGNMENT OF SUTURES WITH SURGICAL NEEDLES”, which is incorporated herein by reference in its entirety.

### TECHNICAL FIELD

The present disclosure generally relates to an exemplary swaging apparatus for attaching a suture to a surgical needle, and in particular a swaging apparatus equipped with an exemplary automatic system for aligning sutures with surgical needles and pull testing swaged surgical needles.

### BACKGROUND

Suturing, an essential and customary part of surgical operations and wound healing, has a history that dates back to ancient times. With the advancements in minimally invasive surgeries (MIS), there has been a significant rise in the use of robotic tools for suturing, which are gradually gaining more autonomy. The process of suturing may refer to a technology where surgical needle is sutured through mechanical interaction, either with fingers or robotic appendages, to physically grasp, push, pull, wiggle or rotate the suture needle. The ability to suture surgical needles using non-contact techniques may substantially reduce the intrusiveness and subsequent trauma associated with certain surgical procedures.

Thus, there is need to engineer automated machines for the assembly and crimping of multivariant surgical sutures, which include a pull test for each suture to ensure a consistently superior quality of thread-needle combination.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more aspects in a simplified form as a prelude to the more detailed description that is presented later. The proper scope of the present disclosure may be ascertained from the claims set forth below in view of the detailed description below and the drawings.

One or more exemplary embodiments describe an exemplary swaging apparatus for attaching an exemplary suture to an exemplary surgical needle. In an exemplary embodiment, an exemplary swaging apparatus may include an exemplary suture guiding assembly. In an exemplary embodiment, an exemplary suture guiding assembly may include an exemplary suture gripping member comprising an exemplary first grip fixedly attached to an exemplary first jaw of an exemplary first parallel gripper through an exemplary fixed end of an exemplary first grip. In an exemplary embodiment, an exemplary suture gripping member may further comprise an exemplary second grip fixedly attached to an exemplary second jaw of an exemplary first parallel gripper through an exemplary fixed end of an exemplary second grip. In an exemplary embodiment, exemplary first and second grips of an exemplary first parallel gripper may be capable of reciprocating away and toward each other in response to an exemplary reciprocating motion of exemplary first and second jaws of an exemplary first parallel gripper in a direction perpendicular to a longitudinal axis of an exemplary suture gripping member. An exemplary suture gripping member may be configured to securely hold at least a portion of an exemplary suture when an inner surface of an exemplary first grip of an exemplary first parallel gripper engages with an inner surface of an exemplary second grip of an exemplary suture gripping member.

In an exemplary embodiment, an exemplary swaging apparatus may further include an exemplary needle management assembly comprising an exemplary needle securing module coaxially disposed opposite to an exemplary suture gripping member. In an exemplary embodiment, an exemplary needle management assembly may comprise an exemplary second parallel gripper and an exemplary clamping member. In an exemplary embodiment, an exemplary second parallel gripper may include an exemplary first jaw fixedly attached, through its front side, to an exemplary first clamp of an exemplary clamping member. In an exemplary embodiment, an exemplary second parallel gripper may further comprise an exemplary second jaw fixedly attached, through its front side, to an exemplary second clamp of an exemplary clamping member. In an exemplary embodiment, exemplary first and second jaws of an exemplary second parallel gripper may be capable of reciprocating in an exemplary axis perpendicular to an exemplary longitudinal axis of an exemplary needle securing module.

In an exemplary embodiment, an exemplary first jaw of an exemplary second parallel gripper may include an exemplary curved groove transversely extending across an exemplary gripping surface thereof, from a top surface of an exemplary first jaw of an exemplary second parallel gripper to an exemplary boundary surface between an exemplary clamping member and an exemplary second parallel gripper. In an exemplary embodiment, an exemplary curved groove may be configured to receive surgical needle when an exemplary gripping surface of an exemplary first jaw of an exemplary second parallel gripper engages with an exemplary gripping surface of an exemplary second jaw of an exemplary second parallel gripper. In an exemplary embodiment, an exemplary curved groove may have a shape and size that may conform with a shape and size of an exemplary curved groove.

In an exemplary embodiment, an exemplary clamping member may further comprise an exemplary swage station configured to receive an exemplary suture-receiving opening of an exemplary surgical needle. In an exemplary embodiment, an exemplary swage station may include an exemplary first slot extending from an exemplary side wall of an exemplary first clamp to an exemplary clamping surface of an exemplary first clamp. In an exemplary embodiment, an exemplary swage station may further comprise an exemplary second slot extending from an exemplary side wall of an exemplary second clamp to an exemplary clamping surface of an exemplary second clamp. In an exemplary embodiment, exemplary first and second slots of an exemplary clamping member may be aligned opposite to each other and both may be parallel to an exemplary boundary surface between an exemplary clamping member and an exemplary second parallel gripper. In an exemplary embodiment, an exemplary swage station may form when an exemplary clamping surface of an exemplary first clamp engages with an exemplary clamping surface of an exemplary second clamp and may be in communication with an exemplary curved groove via an exemplary first hole formed at a centre of an exemplary boundary surface between an exemplary clamping member and an exemplary second parallel gripper.

In an exemplary embodiment, an exemplary clamping member of an exemplary needle securing module may further comprise an exemplary funnel-shaped tipping station. An exemplary needle securing module may be defined by an exemplary first half provided across an exemplary clamping surface of an exemplary first clamp, and an exemplary second half provided across an exemplary clamping surface of an exemplary second clamp. In an exemplary embodiment, an exemplary funnel-shaped tipping station may comprise an exemplary open vertex in communication with an exemplary swage station of an exemplary clamping member through an exemplary second hole. In an exemplary embodiment, an exemplary funnel-shaped tipping station may further comprise an exemplary open base facing toward an exemplary tip of an exemplary suture gripping member. An exemplary funnel-shaped tipping station may be configured to receive an exemplary tip of an exemplary suture through an exemplary open base and align an exemplary tip of an exemplary suture with an exemplary suture-receiving opening of an exemplary surgical needle.

In an exemplary embodiment, an exemplary swaging apparatus may further comprise an exemplary swaging assembly. In an exemplary embodiment, an exemplary swaging assembly may comprise an exemplary first slider actuated by an exemplary first servo motor and an exemplary second slider actuated by an exemplary second servo motor. Exemplary first and second sliders may be arranged coaxially on opposite sides of an exemplary swage station and opposite to one another. In an exemplary embodiment, exemplary first and second sliders may comprise an exemplary first swage die and an exemplary second swage die, respectively. In an exemplary embodiment, exemplary first and second swage dies may face one another and may be configured to mate each other in an exemplary swage station when exemplary first and second sliders linearly reciprocate toward each other. In an exemplary embodiment, exemplary first and second swage dies may be configured to apply force on a threaded end of an exemplary threaded surgical needle in response to a movement created by exemplary first and second servo motors, respectively.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for the purpose of illustration and description only and are not intended as a definition of the limits of the present disclosure. Embodiments of the present disclosure will now be described by way of example in association with the accompanying drawings in which:

FIG. 1A illustrates an isometric view of an exemplary swaging apparatus, consistent with one or more exemplary embodiments of the present disclosure;

FIG. 1B illustrates an exploded view of an exemplary swaging apparatus, consistent with one or more exemplary embodiments of the present disclosure;

FIG. 2 illustrates a rear perspective view of an exemplary suture guiding assembly, consistent with one or more exemplary embodiments of the present disclosure;

FIG. 3A illustrates a rear view of an exemplary gripping member with exemplary first and second grips being in an exemplary non-gripping configuration, consistent with one or more exemplary embodiments of the present disclosure;

FIG. 3B illustrates a rear view of an exemplary gripping member with exemplary first and second grips being in an exemplary gripping configuration, consistent with one or more exemplary embodiments of the present disclosure;

FIG. 4 illustrates a perspective view of an exemplary needle management assembly, consistent with one or more exemplary embodiments of the present disclosure;

FIG. 5 illustrates a perspective view of an exemplary needle securing module, consistent with one or more exemplary embodiments of the present disclosure;

FIG. 6A illustrates an inner side view of an exemplary first jaw and an exemplary first clamp of an exemplary second parallel gripper and an exemplary clamping member, respectively, consistent with one or more exemplary embodiments of the present disclosure;

FIG. 6B illustrates a perspective view of an exemplary first jaw and an exemplary first clamp of an exemplary second parallel gripper and an exemplary clamping member, respectively, consistent with one or more exemplary embodiments of the present disclosure;

FIG. 6C illustrates an inner side view of an exemplary second jaw and an exemplary second clamp of an exemplary second parallel gripper and an exemplary clamping member, respectively, consistent with one or more exemplary embodiments of the present disclosure;

FIG. 7 illustrates a side view of an exemplary second parallel gripper's channel through which an exemplary pressurized air is circulated, consistent with one or more exemplary embodiments of the present disclosure;

FIG. 8 illustrates a perspective view of an exemplary needle management assembly as disassembled from an exemplary needle securing module, consistent with one or more exemplary embodiments of the present disclosure;

FIG. 9A illustrates a side view of an exemplary threaded surgical needle, consistent with one or more exemplary embodiments of the present disclosure;

FIG. 9B illustrates a cross-sectional view of an exemplary suture-receiving opening of an exemplary threaded surgical needle, consistent with one or more exemplary embodiments of the present disclosure;

FIG. 10A illustrates a perspective view of an exemplary swaging assembly, consistent with one or more exemplary embodiments of the present disclosure;

FIG. 10B illustrates a front view of an exemplary swaging assembly, consistent with one or more exemplary embodiments of the present disclosure;

FIG. 11 illustrates a perspective view of exemplary first and second swage dies in a state when an exemplary suture-receiving opening of an exemplary threaded surgical needle is disposed therebetween, consistent with one or more exemplary embodiments of the present disclosure;

FIG. 12 illustrates a perspective view of exemplary first and second swage dies in a state when an exemplary suture-receiving opening of an exemplary threaded surgical needle is under swaging operation, consistent with one or more exemplary embodiments of the present disclosure;

FIG. 13A illustrates a side view of an exemplary swaged surgical needle, consistent with one or more exemplary embodiments of the present disclosure; and

FIG. 13B illustrates a cross-sectional view of an exemplary swaged suture-receiving opening of an exemplary swaged surgical needle, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Disclosed herein is an exemplary swaging apparatus for attaching a suture to a surgical needle. An exemplary swaging apparatus may be beneficial as it may precisely position a tip of an exemplary suture within an exemplary suture-receiving aperture of an exemplary surgical needle. Moreover, an exemplary swaging apparatus may automate an exemplary swaging process, thereby minimizing contamination risks by reducing manual handling of suture and needle. An exemplary swaging apparatus may also incorporate an automated quality control mechanism that may conduct a pulling test on an exemplary swaged surgical needle, ensuring consistent high-quality production of swaged surgical needles. “Swaged needle” may refer to surgical needles where a thread or suture material is directly attached, or swaged, onto a base (i.e., suture-receiving opening) of a needle.

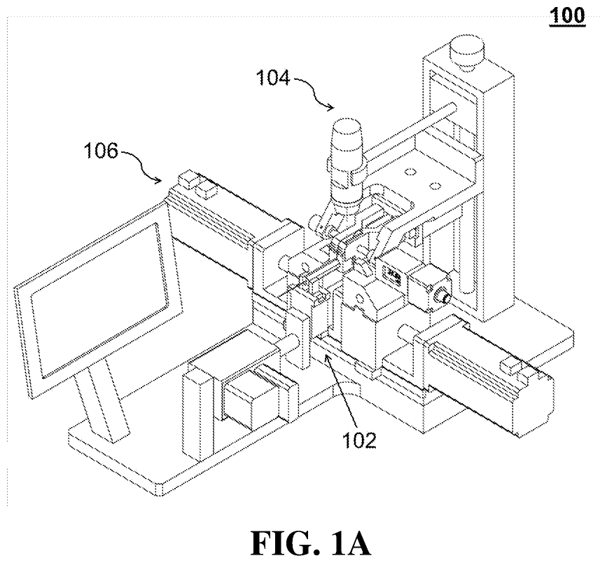

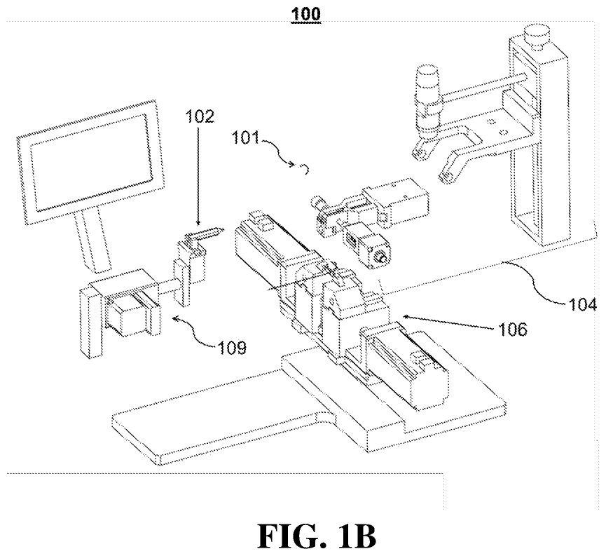

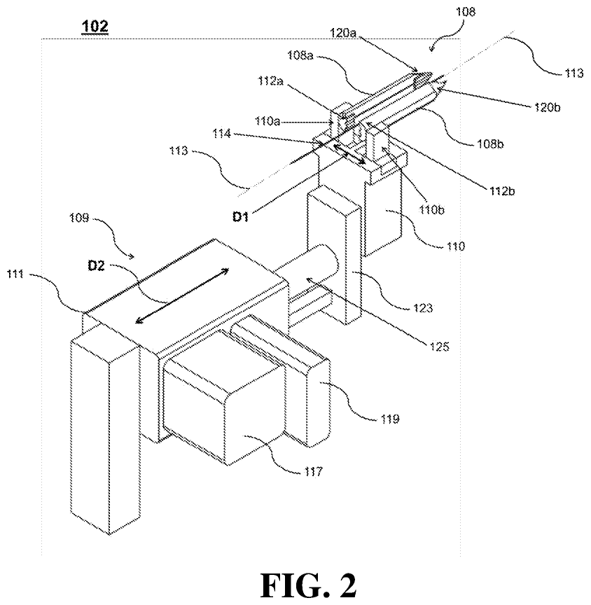

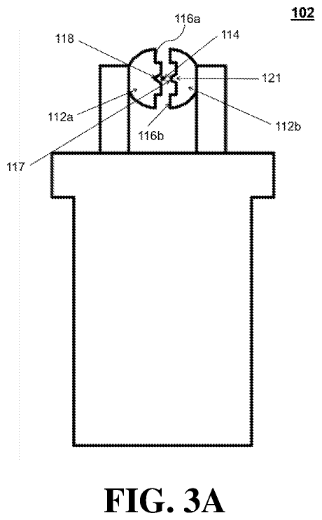

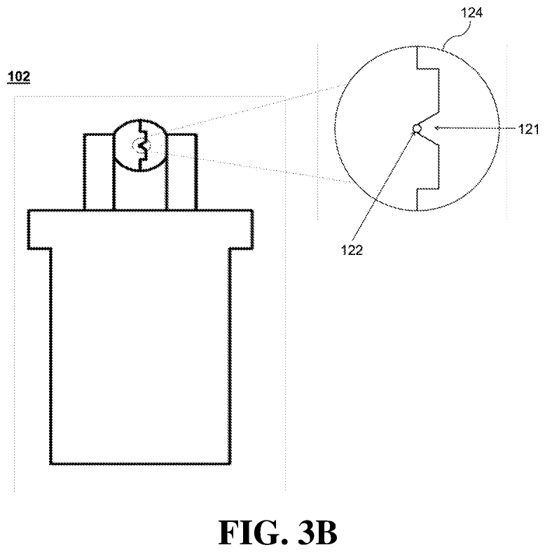

Referring to the figures, FIG. 1A illustrates an isometric view of swaging apparatus 100, consistent with one or more exemplary embodiments of the present disclosure. FIG. 1B illustrates an exploded view of swaging apparatus 100, consistent with one or more exemplary embodiments of the present disclosure. In further detail with respect to FIGS. 1A-B, swaging apparatus 100 may include suture guiding assembly 102, needle management assembly 104, and swaging assembly 106. FIG. 2 illustrates a rear perspective view of suture guiding assembly 102, consistent with one or more exemplary embodiments of the present disclosure. As illustrated in FIG. 2, in an exemplary embodiment, suture guiding assembly 102 may include suture gripping member 108. In an exemplary embodiment, suture gripping member 108 may include first grip 108a and second grip 108b. In an exemplary embodiment, first grip 108a may be fixedly attached to first jaw 110a of first parallel gripper 110 through fixed end 112a of first grip 108a, and second grip 108b fixedly attached to second jaw 110b of first parallel gripper 110 through fixed end 112b of second grip 108b. In an exemplary embodiment, first and second grips (108a and 108b, respectively) of first parallel gripper 110 may be capable of reciprocating away and toward each other in response to an exemplary reciprocating motion of first and second jaws (110a and 110b, respectively) in an exemplary direction perpendicular to longitudinal axis 113 of suture gripping member 108 (i.e., direction D1 as illustrated in FIG. 2). In an exemplary embodiment, suture gripping member 108 may be configured to securely hold at least a portion of suture 114 when inner surface 116a of first grip 108a (shown in FIG. 3A) engages with inner surface 116b of second grip 108b (shown in FIG. 3A). In an exemplary embodiment, details of gripping member 108 are described in context of elements illustrated in FIGS. 3A-B.

FIG. 3A illustrates a rear view of gripping member 108 with first and second grips (108a and 108b, respectively) being in an exemplary non-gripping configuration, consistent with one or more exemplary embodiments of the present disclosure. FIG. 3B illustrates a rear view of gripping member 108 with first and second grips (108a and 108b, respectively) being in an exemplary gripping configuration, consistent with one or more exemplary embodiments of the present disclosure. In an exemplary embodiment, an exemplary non-gripping configuration may refer to a state in which first and second grips (108a and 108b, respectively) of gripping member 108 move apart or disengage. In an exemplary embodiment, an exemplary gripping configuration may refer to a state in which inner surface 116a of first grip 108a engages with inner surface 116b of second grip 108b.

In further detail with respect to FIGS. 3A-B, first grip 108a may comprise groove 118 extending along inner surface 116a of first grip 108a from fixed end 112a of first grip 108a to conical end 120a (shown in FIG. 2) of first grip 108a. In an exemplary embodiment, groove 118 may longitudinally accommodate at least a portion of suture 114. In an exemplary embodiment, second grip 108b of gripping member 108 may include tongue 121 extending along inner surface 116b of second grip 108b from fixed end 112b of second grip 108b to conical end 120b (shown in FIG. 2) of second grip 108b. In an exemplary embodiment, tongue 121 may be aligned with groove 118 such that groove 118 may be capable of receiving tongue 121 when inner surface 116a of first grip 108a engages with inner surface 116b of second grip 108b of gripping member 108. In an exemplary embodiment, as illustrated in FIG. 3B, tongue 121 may further include elongated concavity 122 (shown in enlarged view 124) that may extend along outer edge 117 (shown in FIG. 3A) of tongue 121 and, in turn, an elongated hole may be formed along longitudinal axis 113 of suture gripping member 108 when first and second grips (108a and 108b, respectively) associate with each other.

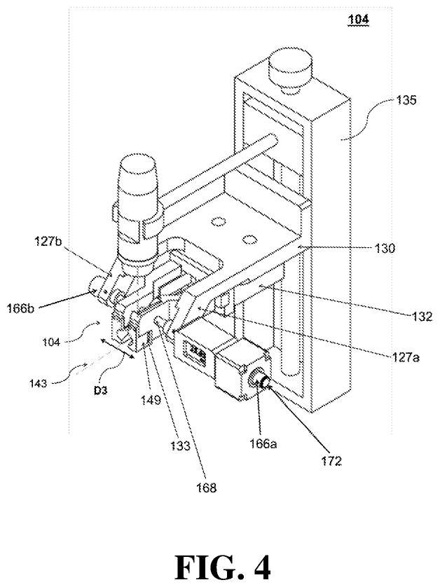

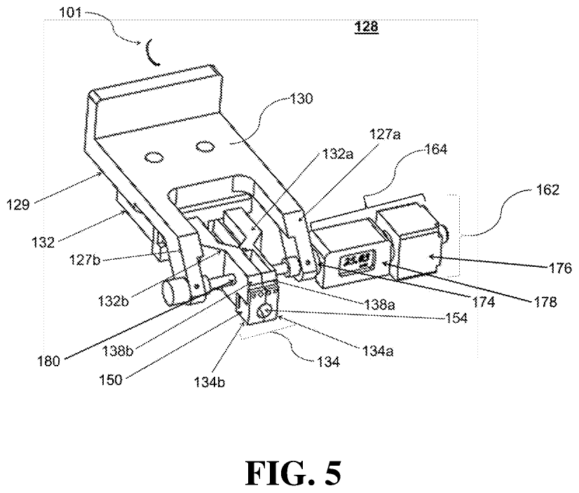

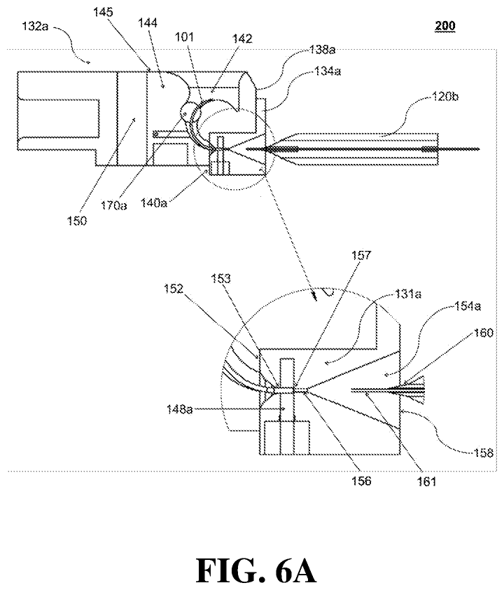

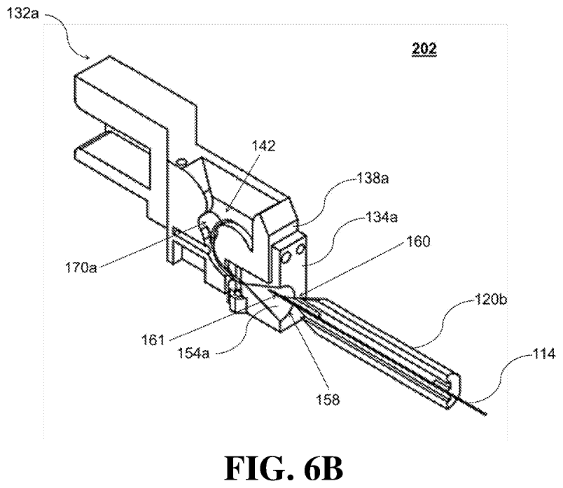

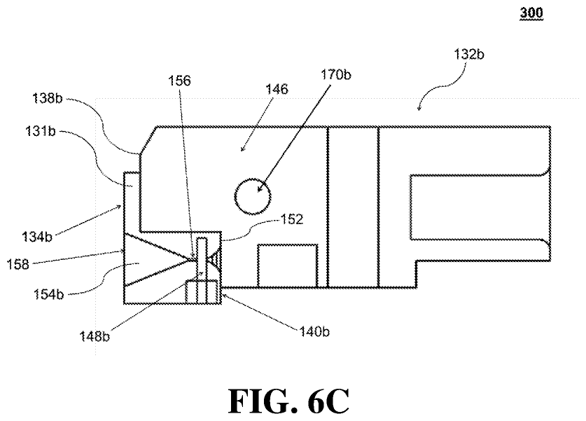

As further shown in FIGS. 1A-B, in an exemplary embodiment, swaging apparatus 100 may further include needle management assembly 104. FIG. 4 illustrates a perspective view of needle management assembly 104, consistent with one or more exemplary embodiments of the present disclosure. As illustrated in FIG. 4 and FIGS. 1A-B, in an exemplary embodiment, needle management assembly 104 may comprise needle securing module 128 disposed opposite to suture gripping member 108. In an exemplary embodiment, needle securing module 128 may be mounted on linear module 135 via holder gripper 130. FIG. 5 illustrates a perspective view of needle securing module 128, consistent with one or more exemplary embodiments of the present disclosure. As illustrated in FIGS. 4-5, in an exemplary embodiment, needle securing module 128 may comprise second parallel gripper 132 and clamping member 134. In an exemplary embodiment, second parallel gripper 132 may include first jaw 132a fixedly attached (through its front side 138a) to first clamp 134a of clamping member 134, and second jaw 132b fixedly attached (through its front side 138b) to second clamp 134b of clamping member 134. In an exemplary embodiment, needle securing module 128 may be coupled to bottom surface 129 of holder gripper 130 such that first and second jaws (132a and 132b, respectively) and clamping member 134 are disposed between first and second arms (127a and 127b, respectively) of holder gripper 130. FIG. 6A illustrates inner side view 200 of first jaw 132a and first clamp 134a of second parallel gripper 132 and clamping member 134, respectively, consistent with one or more exemplary embodiments of the present disclosure. FIG. 6B illustrates perspective view 202 of first jaw 132a and first clamp 134a of second parallel gripper 132 and clamping member 134, respectively, consistent with one or more exemplary embodiments of the present disclosure. FIG. 6C illustrates inner side view 300 of second jaw 132b and second clamp 134b of second parallel gripper 132 and clamping member 134, respectively, consistent with one or more exemplary embodiments of the present disclosure. As illustrated in FIG. 6A, B, and C, front side 138a of first jaw 132a may be attached to rear side 140a of first clamp 134a, and front side 138b of second jaw 132b may be attached to rear side 140b of second clamp 134b. In an exemplary embodiment, first and second jaws (132a and 132b, respectively) of second parallel gripper 132 may be capable of reciprocating in an exemplary axis (labeled as D3 in FIG. 4) perpendicular to longitudinal axis 143 (shown in FIG. 4) of needle securing module 128.

As further shown in FIGS. 6A-B, in an exemplary embodiment, first jaw 132a of second parallel gripper 132 may include curved groove 142 transversely extending across gripping surface 144 of first jaw 132a of second parallel gripper 132, from top surface 145 of first jaw 132a to boundary surface 152 between clamping member 134 and second parallel gripper 132. In an exemplary embodiment, curved groove 142 may be configured to receive surgical needle 101 when gripping surface 144 of first jaw 132a engages with gripping surface 146 of second jaw 132b. In an exemplary embodiment, curved groove 142 may have an exemplary shape and size that may conform with an exemplary shape and size of surgical needle 101.

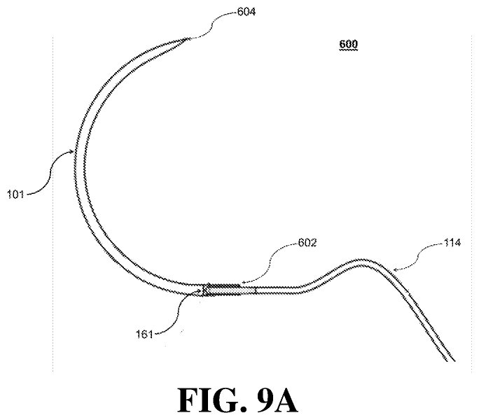

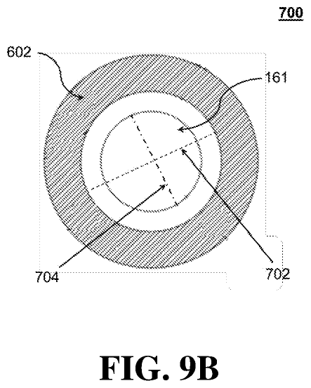

With continued reference to FIG. 4, in an exemplary embodiment, clamping member 134 may further comprise swage station 133 configured to receive suture-receiving opening 602 (shown in FIG. 9A) of surgical needle 101, that may be due to an exemplary structure of swage station 133. In an exemplary embodiment, as illustrated FIG. 6A, B, and C, swage station 133 may include first slot 148a extending from side wall 149 of first clamp 134a (shown in FIG. 4) to clamping surface 131a of first clamp 134a (shown in FIG. 6A). In an exemplary embodiment, swage station 133 may further comprise second slot 148b extending from side wall 150 of second clamp 134b to clamping surface 131b second clamp 134b. In an exemplary embodiment, first and second slots (148a and 148b, respectively) of clamping member 134 may be aligned opposite to each other and both may be parallel to boundary surface 152 between clamping member 134 and second parallel gripper 132. In an exemplary embodiment, swage station 133 may be formed when clamping surface 131a of first clamp 134a engages with clamping surface 131b of second clamp 134b. In an exemplary embodiment, swage station 133 may refer to an exemplary empty space extending from side wall 149 of first clamp 134a to side wall 150 of second clamp 134b, proximate to boundary surface 152 between clamping member 134 and second parallel gripper 132. In an exemplary embodiment, swage station 133 may be in communication with curved groove 142 via first hole 153 formed at a centre of boundary surface 152 between clamping member 134 and second parallel gripper 132. In an exemplary embodiment, first hole 153 may aid in receiving suture-receiving opening 602 of surgical needle 101 from curved groove 142 into swage station 133.

With further reference to FIGS. 6A, 6B, and 6C, clamping member 134 of needle securing module 128 may further include funnel-shaped tipping station 154 (shown in FIG. 5) defined by first half 154a and second half 154b. In an exemplary embodiment, first half 154a may be disposed across clamping surface 131a of first clamp 134a, and second half 154b may be disposed across clamping surface 131b of second clamp 134b. In an exemplary embodiment, funnel-shaped tipping station 154 may comprise open vertex 156 that may be in communication with swage station 133 through second hole 157 formed at a centre of boundary surface 155 between funnel-shaped tipping station 154 and swage station 133. In an exemplary embodiment, first and second holes (153 and 157, respectively) may be aligned concentrically opposite to each other. Meanwhile, in an exemplary embodiment, first and second holes (153 and 157, respectively) may be aligned with open vertex 156.

In an exemplary embodiment, funnel-shaped tipping station 154 may further include open base 158 facing toward tip 160 (shown in FIGS. 6A-B) of suture gripping member 108. In an exemplary embodiment, funnel-shaped tipping station 154 may be configured to receive tip 161 (shown in FIGS. 6A-B) of suture 114 and align tip 161 of suture 114 with suture-receiving opening 602 of surgical needle 101, that may be due to conical structure of funnel-shaped tipping station 154 with its open vertex 156 facing toward swage station 133 where suture-receiving opening 602 of surgical needle 101 is parked. In an exemplary embodiment, second hole 157 and open vertex 156 may have a same diameter that may be less than a diameter of suture-receiving opening 602 of surgical needle 101.

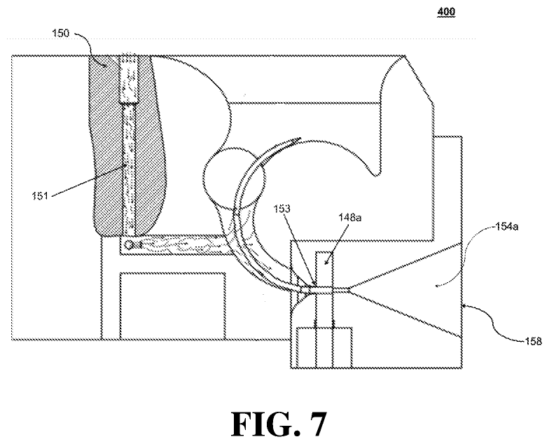

As further shown in FIG. 6A-B, in an exemplary embodiment, second parallel gripper 132 may further include channel 150 that may transversely extend across first jaw 132a from top surface 145 of first jaw 132a to curved groove 142. In an exemplary embodiment, channel 150 may allow circulation of pressurized air 151 (see FIG. 7) through channel 150 and at least a portion of curved groove 142. FIG. 7 illustrates side view 400 of second parallel gripper's 132 channel 150 through which pressurized air 151 is circulated, consistent with one or more exemplary embodiments of the present disclosure. As depicted in FIG. 7, in an exemplary embodiment, pressurized air 151 may facilitate swift and smooth transition of suture-receiving opening 602 of surgical needle 101 toward first hole 153 of swage station 133. Moreover, pressurized air 151 may be utilized to purge channel 150 from contaminants and debris. In an exemplary embodiment, an exemplary air compressor producing pressurized air 151 may maintain pressure of pressurized air 151 at about 6 bar. In an exemplary embodiment, to guide pressurized air 151 within channel 150, an exemplary design of channel 150 may be such that it prevents vortex flow within channel 150, allowing for a gentle force to be exerted on a center mass of surgical needle 101.

With further reference to FIGS. 4-5, needle management assembly 104 may further comprise needle fixing mechanism 162. In an exemplary embodiment, needle fixing mechanism 162 may include stabilizing differential system 164 and exemplary pair of rods (166a and 166b) coaxially arranged on opposite sides of second parallel gripper 132 and opposite to each other. In an exemplary embodiment, exemplary pair of rods (166a and 166b) may comprise reciprocating rod 166a with inner end 168 (shown in FIG. 4) extending outwardly from an inner side of first arm 127a to first jaw 132a of second parallel gripper 132. In an exemplary embodiment, inner end 168 of reciprocating rod 166a may pass into first aperture 170a (shown in FIGS. 6A-B) that may be provided through first jaw 132a of second parallel gripper 132. In an exemplary embodiment, reciprocating rod 166a may further include outer end 172 (shown in FIG. 4) extending outwardly from an outer side of first arm 127a. In an exemplary embodiment, reciprocating rod 166a may carry stabilizing differential system 164 between outer end 172 of reciprocating rod 166a and attaching point 174 (shown in FIG. 5) of reciprocating rod 166a to outer side of first arm 127a. In an exemplary embodiment, stabilizing differential system 164 may include stepper motor 176 and dimensional indicator 178 both of which may be mounted on reciprocating rod between outer end 172 and attaching point 174.

In an exemplary embodiment, pair of rods (166a and 166b) may further include fixed rod 166b with inner end 180 (shown in FIG. 5) extending outwardly from an inner side of second arm 127b to second jaw 132b of second parallel gripper 132. In an exemplary embodiment, inner end 180 of fixed rod 166b may pass into second aperture 170b (shown in FIG. 6C) provided through second jaw 132b of second parallel gripper 132. In an exemplary embodiment, first and second apertures (170a and 170b, respectively) may be disposed coplanarly on first jaw 132a and second jaw 132b, respectively, and may be in communication with a central zone of curved groove 142. In an exemplary embodiment, subsequent to inserting surgical needle 101 into curved groove 142 of second parallel gripper 132, reciprocating rod 166a may be reciprocated along first aperture 170a by stepper motor 176 so that inner end 168 of reciprocating rod 166a coincides with surgical needle's 101 body, and surgical needle 101 is securely positioned between inner end 168 of reciprocating rod 166a and inner end 180 of fixed rod 166b. In an exemplary embodiment, stabilizing differential system 164 may further include dimensional indicator 178 that may be used for measuring thickness of suture-receiving opening 602 of surgical needle 101 before fixing surgical needle 101 between inner end 168 of reciprocating rod 166a and inner end 180 of fixed rod 166b.

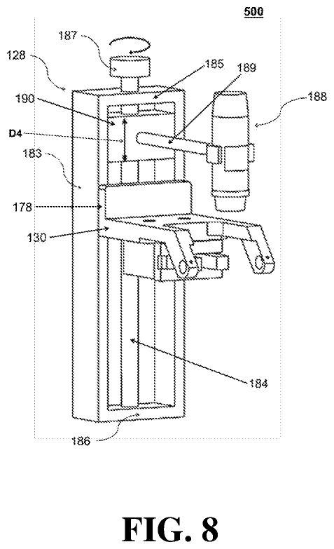

FIG. 8 illustrates perspective view 500 of needle management assembly 104 as disassembled from needle securing module 128, consistent with one or more exemplary embodiments of the present disclosure. In further detail with respect to FIG. 8, linear module 128 of needle management assembly 104 may include body 183 in which slider screw 184 may extend from upper wall 185 to bottom wall 186 of body 183. In an exemplary embodiment, slider screw 184 may be rotated using handle 187. In an exemplary embodiment, holder gripper 130 may be coupled to body 183 between upper wall 185 and bottom wall 186 via its rear edge 178. As further shown in FIG. 8, in an exemplary embodiment, needle management assembly 104 may further include vision system 188 that may be slidably mounted on slider screw 184 by slider 190 such that vision system 188 faces toward needle securing module 128 to image surgical needle 101, with respect to orientation and position of surgical needle 101, before and after swaging. In an exemplary embodiment, vision system 188 may be attached to and spaced apart from slider 190 by shank 189 disposed between vision system 188 and slider 190. In an exemplary embodiment, slider 190 may be moved in D4 direction (shown in FIG. 8) by rotating handle 187. In an exemplary embodiment, by manipulating slider 190 within its range of motion (in D4 direction), vision system's 188 field of view may be adjusted to directly focus on surgical needle 101.

FIG. 9A illustrates a side view of threaded surgical needle 600, consistent with one or more exemplary embodiments of the present disclosure. Threaded surgical needle 600 may refer to a state in which tip 161 of suture 114 is inserted into suture-receiving opening 602 of surgical needle 101. As depicted in FIG. 9A, surgical needle 101 may include pointed tip 604 and suture-receiving opening 602 capable of receiving tip 161 of suture 114. FIG. 9B illustrates cross-sectional view 700 of suture-receiving opening 602 of threaded surgical needle 600, consistent with one or more exemplary embodiments of the present disclosure. In further detail with respect to FIG. 9B, suture-receiving opening 602 may have a diameter (labeled as 702) greater than a diameter (labeled as 704) of suture 114. In an exemplary embodiment, diameter 702 of suture-receiving opening 602 may range between 0.15 mm and 1.27 mm, contingent on an exemplary size of surgical needle 101.

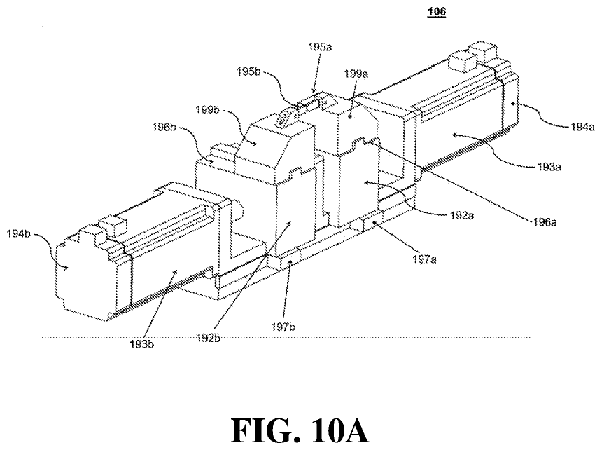

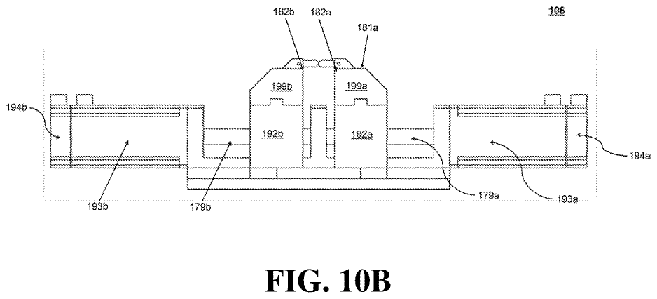

With further reference to FIGS. 1A-B, swaging apparatus 100 may further comprise swaging assembly 106. FIG. 10A illustrates a perspective view of swaging assembly 106, consistent with one or more exemplary embodiments of the present disclosure. FIG. 10B illustrates a front view of swaging assembly 106, consistent with one or more exemplary embodiments of the present disclosure. In an exemplary embodiment, swaging assembly 106 may include first slider 192a actuated by first servo motor 193a and second slider 192b actuated by second servo motor 193b. In an exemplary embodiment, as illustrated in FIG. 1A and FIGS. 10A-B, first and second sliders (192a and 192b, respectively) may be arranged coaxially on opposite sides of swage station 133 and opposite to each other. In an exemplary embodiment, first and second sliders (192a and 192b, respectively) may further include first and second rotary encoders (194a and 194b, respectively) attached to an outer side of first and second servo motors (193a and 193b, respectively), respectively.

As further shown in FIGS. 10A-B, first and second sliders (192a and 192b, respectively) may further comprise first swage die 195a and second swage die 195b, respectively, provided on top surface 196a of first slider 192a and top surface 196b of second slider 192b, respectively. In an exemplar embodiment, first and second swage dies (195a and 195b, respectively) may be attached to first and second sliders (192a and 192b, respectively) via first adjuster 199a and second adjuster 199b, respectively. In an exemplary embodiment, first and second adjusters (199a and 199b, respectively) may be attached to top surface 196a of first slider 192a and top surface 196b of second slider 192b, respectively. In an exemplary embodiment, first swage die 195a may be attached to inner edge 182a of top surface 181a of first adjuster 199a, and second swage die 195b may be attached to inner edge 182b of top surface 181b of second adjuster 199b. In an exemplary embodiment, first and second adjusters (199a and 199b, respectively) may be used to align first and second swage dies (195a and 195b, respectively) to be coaxially disposed opposite to each other.

In an exemplary embodiment, first and second swage dies (195a and 195b, respectively) may face one another and may mate each other in swage station 133 when first and second sliders (192a and 192b, respectively) linearly slide toward each other. In an exemplary embodiment, first and second swage dies (195a and 195b, respectively) may aid in applying force on suture-receiving opening 602 of threaded surgical needle 600 (shown in FIGS. 9A-B) in response to a movement created by first and second servo motors (193a and 193b, respectively). In an exemplary embodiment, first and second sliders (192a and 192b, respectively) may further include first and second linear encoders (197a and 197b, respectively) coaxially attached to bottom side of first slider 192a and bottom side of second slider 192b, respectively. In an exemplary embodiment, first and second linear encoders (197a and 197b, respectively) may be employed to ensure precise control and to implement a fully closed-loop control system during an exemplary swaging process. In an exemplary embodiment, an accurate positioning of first and second sliders (192a and 192b, respectively) may be relayed to an exemplary microprocessor by both first and second linear encoders (197a and 197b, respectively), as well as first and second rotary encoders (194a and 194b, respectively). In an exemplary embodiment, both of first and second servo motors (193a and 193b, respectively) may be regulated using an exemplary full closed-loop method to apply a completely synchronized force on suture-receiving opening 602 of surgical needle 101.

In an exemplary embodiment, as illustrated in FIG. 10B, swaging assembly 106 may further include first screw 179a attaching first slider 192a to first servo motor 193a, and second screw 179b attaching second slider 192b to second servo motor 193b. In an exemplary embodiment, first and second screws (179a and 179b, respectively) may aid in transmitting first and second servo motors' (193a and 193b, respectively) rotational force to first slider 192a and second slider 192b, as well as transforming this rotational force into linear motion. In an exemplary embodiment, an exemplary design of first screw 179a may be such that one complete rotation of each of first and second servo motors (193a and 193b, respectively) may result in a linear movement of about 5 mm. In an exemplary embodiment, first and second screws (179a and 179b, respectively) may also serve to transfer an exemplary required force for swaging operations from first and second servo motors (193a and 193b, respectively) to first and second sliders (192a and 192b, respectively).

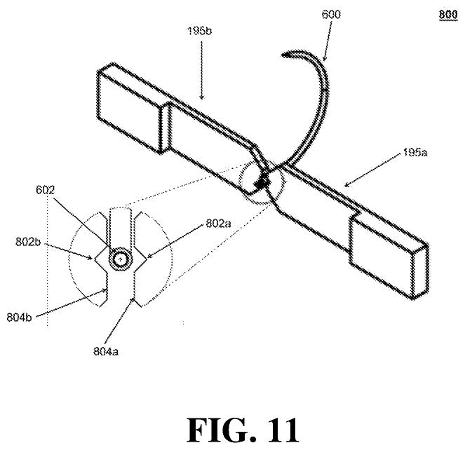

FIG. 11 illustrates perspective view 800 of first and second swage dies (195a and 195b, respectively) in a state when suture-receiving opening 602 of threaded surgical needle 600 is disposed therebetween, consistent with one or more exemplary embodiments of the present disclosure. In further detail with respect to FIG. 11, both first swage die 195a and second swage die 195b may simultaneously move towards each other during swaging process, consequently compressing suture-receiving opening 602 of threaded surgical needle 600 for creating an attachment between suture-receiving opening 602 and tip 161 of suture 114. As depicted in FIG. 11, in an exemplary embodiment, first swage die 195a may include first semi-square groove 802a disposed at a centre of peripheral edge 804a of first swage die 195a, and second swage die 195b may include second semi-square groove 802b disposed at a centre of peripheral edge 804b of second swage die 195b. In an exemplary embodiment, first and second semi-square grooves (802a and 802b, respectively) may accommodate suture-receiving opening 602 of threaded surgical needle 600.

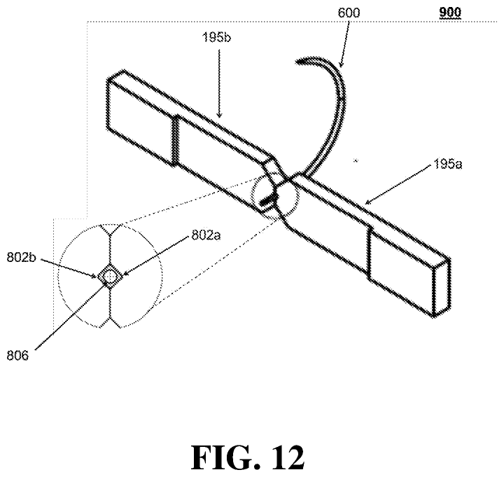

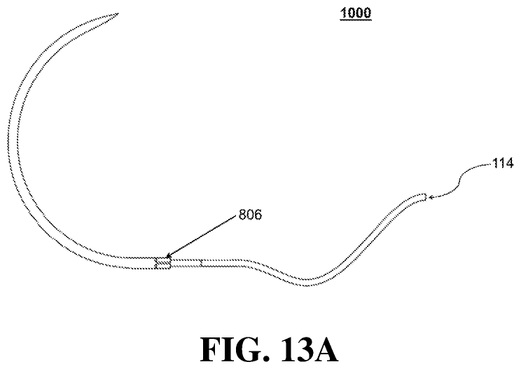

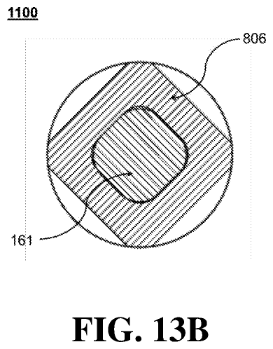

FIG. 12 illustrates perspective view 900 of first and second swage dies (195a and 195b, respectively) in a state when suture-receiving opening 602 of threaded surgical needle 600 is under swaging operation, consistent with one or more exemplary embodiments of the present disclosure. In further detail with respect to FIG. 12, first and second swage dies (195a and 195b, respectively) may operate in unison. In an exemplary embodiment, first and second swage dies (195a and 195b, respectively) may move towards each other and subsequently compress suture-receiving opening 602 of threaded surgical needle 600. This operation (i.e., compression) may result in deformation of suture-receiving opening 602 and formation of swaged suture-receiving opening 806. In an exemplary embodiment, it may be possible for tip 161 of suture 114, disposed inside suture-receiving opening 602, to establish an attachment with inner wall of suture-receiving opening 602 due to a friction between tip 161 of suture 114 and inner wall of suture-receiving opening 602. In an exemplary embodiment, upon compression of suture-receiving opening 602, swaging surface 804a (shown in FIG. 11) of first swage die 195a may associate with swaging surface 804b (shown in FIG. 11) of second swage dies (195a and 195b, respectively) may associate with each other. FIG. 13A illustrates a side view of swaged surgical needle 1000, consistent with one or more exemplary embodiments of the present disclosure. FIG. 13B illustrates a cross-sectional view 1100 of swaged suture-receiving opening 806 of swaged surgical needle 1000, consistent with one or more exemplary embodiments of the present disclosure.

Referring back to FIG. 2, in an exemplary embodiment, suture guiding assembly may further include pull-test mechanism 109 which may pull test swaged surgical needle 1000 (shown in FIG. 13A) to ensure that the minimum and/or destructive pull-test requirements of surgical operations are met. In an exemplary embodiment, pull-test mechanism 109 may include linear actuator 111 that may be capable of creating motion in a straight line similar to an exemplary direction of D2 (shown in FIG. 2). It may be understood that, in an exemplary embodiment, linear actuator 111 may include servo motor 117 attached thereto. “Servo motor” may refer to a mechanical device used in linear actuators that comprise sensors for monitoring speed and position of linear actuator and make necessary adjustments to reach an ideal performance. Moreover, it may be understood that linear actuator 111 may further comprise controller 119 attached thereto. “linear actuator controller” may refer to a mechanical device that may manage transmutation of energy—typically electrical—into linear motions including aspects such as the speed, direction, and position of an actuator's movements. In an exemplary embodiment, pull-test mechanism 109 may further include load cell 123 disposed between first parallel gripper 110 and linear actuator 111. In an exemplary embodiment, load cell 123 may be attached to linear actuator 111 via shaft 125. It may be understood that “load cell” (e.g., load cell 123) may refer to an exemplary electro-mechanical senor used to measure force or weight.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

1. A swaging apparatus for attaching a suture to a surgical needle, comprising:
a suture guiding assembly comprising:
a suture gripping member comprising a first grip fixedly attached to a first jaw of a first parallel gripper through a fixed end of the first grip, and a second grip fixedly attached to a second jaw of the first parallel gripper through a fixed end of the second grip, wherein the first and second grips of the first parallel gripper are capable of reciprocating away from and toward each other in response to a reciprocating motion of the first and second jaws of the first parallel gripper in a direction perpendicular to a longitudinal axis of the suture gripping member, wherein the suture gripping member is configured to securely hold at least a portion of the suture when an inner surface of the first grip of the first parallel gripper engages with an inner surface of the second grip of the suture gripping member; a needle management assembly comprising:
a needle securing module coaxially disposed opposite to the suture gripping member and comprising a second parallel gripper and a clamping member, the second parallel gripper comprising a first jaw fixedly attached, through its front side, to a first clamp of the clamping member, the second parallel gripper further comprising a second jaw fixedly attached, through its front side, to a second clamp of the clamping member, wherein the first and second jaws of the second parallel gripper are capable of reciprocating in an axis perpendicular to a longitudinal axis of the needle securing module;
wherein the first jaw of the second parallel gripper comprises a curved groove transversely extending across a gripping surface thereof, from a top surface of the first jaw of the second parallel gripper to a boundary surface between the clamping member and the second parallel gripper, wherein the curved groove is configured to receive the surgical needle when the gripping surface of the first jaw of the second parallel gripper engages with a gripping surface of the second jaw of the second parallel gripper, wherein the curved groove has a shape and size that conforms with a shape and size of the surgical needle;
wherein the clamping member further comprises a swage station configured to receive suture-receiving opening of the surgical needle, the swage station comprising a first slot extending from a side wall of the first clamp to a clamping surface of the first clamp, the swage station further comprising a second slot extending from a side wall of the second clamp to a clamping surface of the second clamp, wherein the first and second slots of the clamping member are aligned opposite to each other and both are parallel to the boundary surface between the clamping member and the second parallel gripper, wherein the swage station forms when the clamping surface of the first clamp engages with the clamping surface of second clamp, wherein the swage station is in communication with the curved groove via a first hole formed at a centre of the boundary surface between the clamping member and the second parallel gripper;
wherein the clamping member of the needle securing module further comprises a funnel-shaped tipping station defined by a first half provided across the clamping surface of the first clamp, and a second half provided across the clamping surface of the second clamp, wherein the funnel-shaped tipping station comprises an open vertex in communication with the swage station of the clamping member through a second hole formed at a centre of a boundary surface between the funnel-shaped tipping station and the swage station, the funnel-shaped tipping station further comprising an open base facing toward a tip of the suture gripping member, the funnel-shaped tipping station configured to receive a tip of the suture through the open base and align the tip of the suture with the suture-receiving opening of the surgical needle; and
a swaging assembly comprising a first slider actuated by a first servo motor and a second slider actuated by a second servo motor, the first and second sliders arranged coaxially on opposite sides of the swage station and opposite to one another, wherein the first and second sliders comprise a first swage die and a second swage die, respectively, the first and second swage dies face one another and are configured to mate each other in the swage station when the first and second sliders linearly reciprocate toward each other, and the first and second swage dies are configured to apply force on a threaded end of a threaded surgical needle in response to a movement created by the first and second servo motors, respectively.

2. The swaging apparatus of claim 1, wherein the gripping member comprises:
a groove extending along the inner surface of the first grip of the gripping member from the fixed end of the first grip to a conical end of the first grip, the groove configured to longitudinally accommodate the at least a portion of the suture; and
a tongue extending along the inner surface of the second grip of the gripping member from the fixed end of the second grip to a conical end of the second grip, wherein the tongue is aligned with the groove such that the groove is capable of receiving the tongue when the inner surface of the first grip engages with the inner surface of the second grip of the gripping member.

3. The swaging apparatus of claim 1, wherein the needle management assembly further comprises a needle fixing mechanism, the needle fixing mechanism comprising:
a stabilizing differential system; and
a pair of rods coaxially arranged on opposite sides of the second parallel gripper and opposite to each other, the pair of rods comprising a reciprocating rod with an inner end passing into a first aperture provided through the first jaw of the second parallel gripper, the pair of rods further comprising a fixed rod with an inner end passing into a second aperture provided through the second jaw of the second parallel gripper, the fixed rod disposed coaxially opposite to the reciprocating rod and perpendicular to the second jaw of the second parallel gripper.

4. The swaging apparatus of claim 3, wherein the reciprocating rod carries the stabilizing differential system.

5. The swaging apparatus of claim 3, wherein the stabilizing differential system comprises a stepper motor and a dimensional indicator.

6. The swaging apparatus of claim 3, wherein the first and second apertures are disposed coplanarly on the first and second jaws of the second parallel gripper, respectively, and are in communication with a central zone of the curved groove.

7. The swaging apparatus of claim 1, wherein the second parallel gripper further comprises a channel transversely crossing the gripping surface of the first jaw of the second parallel gripper from the top surface of the first jaw of the second parallel gripper to the curved groove.

8. The swaging apparatus of claim 7, wherein the channel is configured to allow circulation of a pressurized air through the channel and at least a portion of the curved groove.
